(19) United States
(12) Patent Application Publication
(10) Pub. No.: US 2004/0049229 A1
(43) Pub. Date: Mar. 11, 2004

(54) Apparatus and method for genetic and biological enhancement utilizing a radio frequency, broadspectrum to enhance cellular mitosis and to increase vascular dialation by the increase of cellular transfer rates

(76) Inventor: Todd Taricco, Zephyr Cove, NV (US)

Correspondence Address: IRELL & MANELLA LLP, 840 NEWPORT CENTER DRIVE, SUITE 400, NEWPORT BEACH, CA, 92660, US

(21) Appl. No.: 10/236,516

(22) Filed: Sep. 6, 2002

(51) Int. Cl. A61N 1/00

(52) U.S. Cl. 607/1

(57) ABSTRACT

A medical method for treating an organic substance that includes applying a broad spectrum of radio frequency energy to the substance.

### BACKGROUND OF THE INVENTION

1. Field of the Invention

The subject matter disclosed generally relates to the field of treating an organic substance such as a cell with radio frequency energy.

2. Background Information

There have been various medical procedures that implement electrical or electro-magnetic energy. For example, chemotherapy techniques employ the bombardment of cancerous tissue with electro-magnetic energy in the x-ray frequency range. The x-ray energy destroys the cancerous tissue.

There have also been developed procedures to stimulate cell production using microwave frequency energy. For example, there is a procedure wherein energy is directed toward tissue to stimulate DNA reproduction. The frequency of the microwave energy wave is selected to stimulate the reproduction of DNA under the given conditions of the procedure. Such a technique requires extensive empirical analysis to determine the correct frequency for each particular tissue. Additionally, the apparatus and environmental control system required to properly stimulate DNA reproduction with the technique is relatively expensive and would be cost prohibitive for most medical service providers. It would be desirable to provide a relatively inexpensive system that can stimulate cell division.

### BRIEF SUMMARY OF THE INVENTION

A medical method for treating an organic substance that includes applying a broad spectrum of radio frequency energy to the substance.

### BRIEF DESCRIPTION OF THE DRAWINGS

FIGS. 1 is a schematic of an apparatus that can apply a broad spectrum of radio frequency energy to an organic substance;

FIG. 2 is a graph showing a waveform created by the apparatus;

FIG. 3 is an illustration showing the broad spectrum of radio frequency being applied to a medical device;

FIG. 4 is a schematic of an alternate embodiment of the apparatus;

FIG. 5 is a schematic of an embodiment of an apparatus for emitting RF energy into tissue.

### DETAILED DESCRIPTION

Disclosed is a medical method for treating an organic substance such as a cell. The method includes applying a broad spectrum of radio frequency energy to the organic substance. The broad spectrum includes signals extending across a range of RF frequencies. By way of example, the organic substance may be tissue wherein the RF energy stimulates cell reproduction.

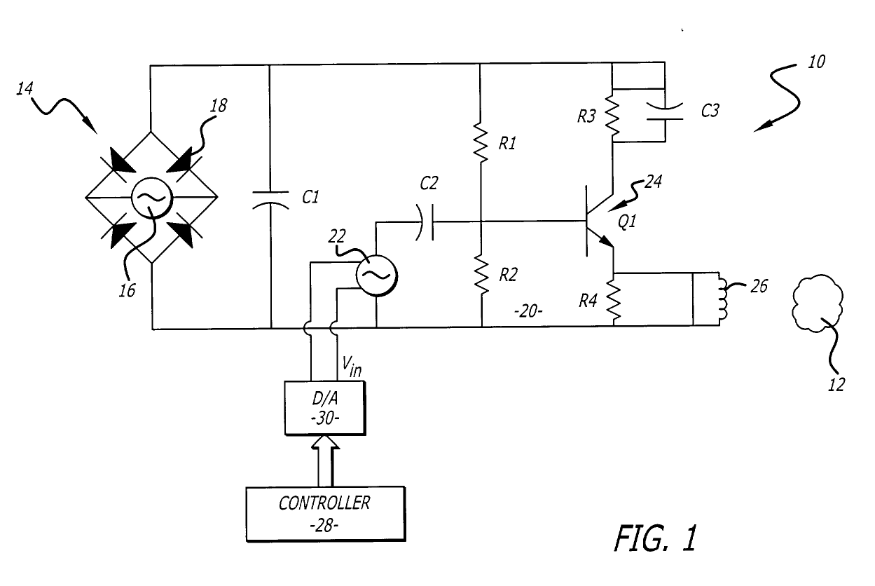

Referring to the drawings more particularly by reference numbers, FIG. 1 shows an apparatus 10 for applying a broad spectrum of radio frequency energy to an organic substance 12. The apparatus may include a full wave rectifier 14 that converts AC power from a power source 16 to a DC voltage level. The rectifier 14 may include a plurality of diodes 18 that are coupled to an output capacitor C₁. The output capacitor C₁ is coupled to a modulation circuit 20. The modulation circuit 20 modulates the DC voltage provided by the rectifier 14. Although a rectifier 14 is shown and described, it is to be understood that the modulation circuit 20 may modulate an AC signal.

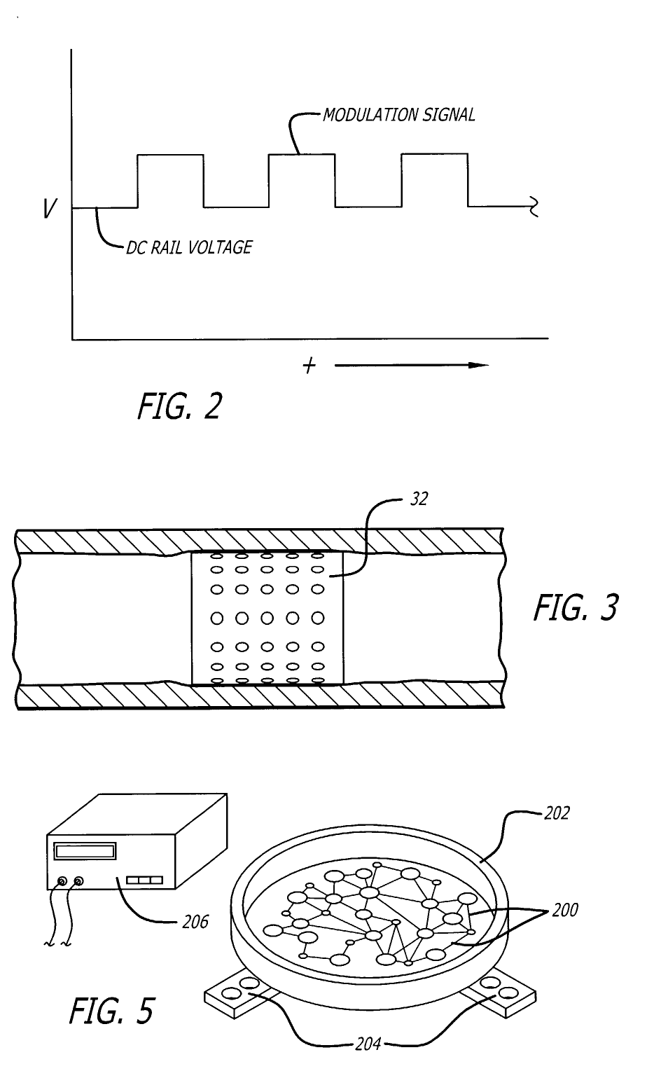

FIG. 2 shows a waveform that can be applied to the organic substance. The waveform may include a series of pulses modulated onto the DC signal. The frequency and width of the pulses can be varied by the modulation circuit 20. The frequency of the pulses is in the radio frequency (RF) range between 50 kilohertz (Khz) and 200 megahertz (Mhz). The modulation circuit 20 generates a number of pulses across a broad spectrum of the RF range.

The broad spectrum signal may be created by generating a sequence of pulses, wherein each set of pulses in the sequence has a different frequency. For example, a series of pulses having a frequency of 50 Khz may be generated followed by a series of pulses at 52.5 Khz and so forth and so on. Although an orderly sequence of pulses is described, it is to be understood that the modulation circuit 20 may generate a random or pseudo-random series of pulses having varying frequencies. Alternatively, the modulation circuit 20 may be replaced with a noise generator that generates electrical noise across the RF frequency spectrum.

Referring to FIG. 1, the modulation circuit 20 may include an oscillator 22 that drives a switch 24. The oscillator 22 may be a voltage controlled variable waveform generator. By way of example, the oscillator 22 may produce a square wave or a sawtooth waveform.

The switch 24 may be a common collector emitter follower circuit which contains an npn bipolar junction transistor Q₁. The base junction of the transistor Q₁ is connected to DC biasing resistors R₁ and R₂. The biasing resistors R₁ and R₂ are coupled to the oscillator 22 by a by-pass capacitor C₂. The collector junction of the transistor Q₁ is connected to a collector resistor R₃ and a by-pass capacitor C₃. The emitter junction of the transistor Q₁ is connected to a emitter resistor R₄. The emittor resistor R₄ is coupled to a transmitter 26.

The transmitter 26 may be an antenna that emits electro-magnetic waves which are applied to the organic substance 12. Alternatively, the transmitter 26 may be an electrode that passes current directly through the organic substance 12, or other means for applying the broad spectrum radio frequency signal to the substance 12.

The oscillator 22 may be coupled to a controller 28 by a digital to analog converter (ADC) 30. The controller 28 provides output commands to vary the frequency of the oscillator 22 and the waveform of the modulation circuit 20. The ADC 30 converts the binary output of the controller 28 to an analog signal. The frequency of the analog signal varies in accordance with the binary output of the controller 28. The controller 28 operates in accordance with a software/firmware routine that generates a frequency sweep. The frequency sweep may include a series of output commands from the controller 28 to continuously vary the output frequency of the modulation circuit across the RF frequency spectrum.

In an experiment where a broad spectrum of RF incubated cells (A+T-20 cells) that make and store beta-endorphins within secretory granules there was observed a 2× increase in the level of beta-endorphin and a decrease in the level of beta-LPH. It is believed that the beta-LPH is processed into beta-endorphins by a molecular scissors known as proprotein convertase.

The organic substance may be human tissue, wherein the application of the broad spectrum RF signal induces mitosis and cell division. It is believed that the broad spectrum RF signal may be used to generate peptide hormones such as beta-endorphins. The broad spectrum RF signal may also enhance blood flow. This procedure may be used to stimulate stem cell reproduction.

FIG. 3 shows an application for using the apparatus 10. A medical device 32 may be implanted into a patient. By way of example, the medical device 32 may be a stent attached to an arterial wall. The device 32 is typically constructed from a metal material that will receive electro-magnetic waves transmitted by the apparatus 10. The apparatus 10 transmits the broad spectrum RF signal which is received by the medical device 32. The medical device 32 is excited by the electro-magnetic signal and transfers the RF energy into the adjoining tissue and blood passing through the artery. The medical device 32 functions as a focusing element for the RF energy transmitted by the apparatus 10. The transfer of RF energy may also induce cell division of the adjacent tissue to enhance tissue attachment to the device 32.

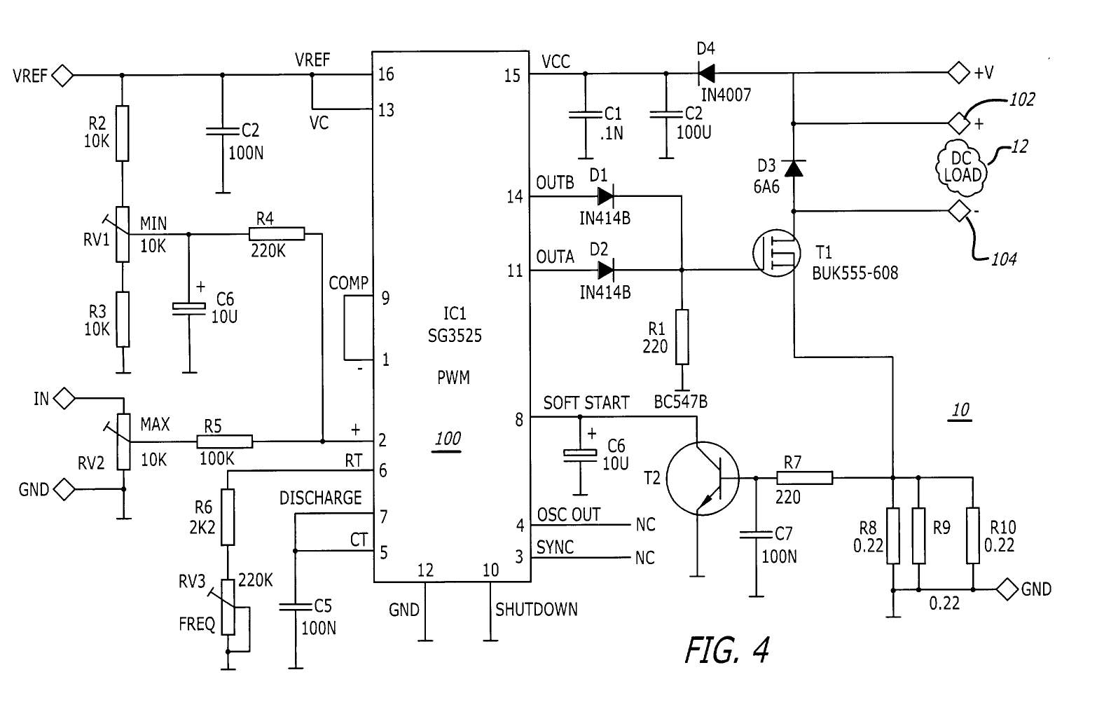

FIG. 4 shows an alternate embodiment of the apparatus 10′. The apparatus 10′ is a motor control circuit modified to operate at a speed that will generate noise across the RF frequency spectrum. The apparatus 10′ includes a pulse width modulator 100 connected to a transistor T₁ by a pair of diodes D₁ and D₂ and resistor R₁. The transistor T₁ functions as a switch for a signal applied to the organic substance 12. The voltage across the organic substance is limited by diode D₃. The Vcc input to the modulator 100 may be connected to a diode D₄ and filtering capacitors C₁ and C₂. The Vref pin of the modulator 100 may be connected to a RC circuit containing resistors R₂, R₃ and R₄, capacitors C₃ and C₄ and a variable resistor Rᵥ₁. The + pin of the controller may be connected to resistor R₅ and variable resistor Rᵥ₂. The variable resistor Rᵥ₂ may be connected to an input pin IN.

The internal clock of the controller 100 may be connected to resistor R₆, capacitor C₅ and a variable resistor Rᵥ₃. The apparatus may include a soft start feature defined by transistor T₂, capacitors C₆ and C₇, and resistors R₇, R₈, R₉ and R₁₀.

The modulator 100 may generate a plurality of output signals at pins 11 and 14. The output signals switch the transistor T₁ between on and off states to create a series of pulses. The output signals vary so that the apparatus creates a series of pulses that vary across the RF frequency spectrum. The current may flow directly through the tissue 12 between electrodes 102 and 104.

FIG. 5 shows an embodiment for applying the RF energy to tissue. A conductive wire 200 is routed along the bottom of a petri dish 202. The wire 200 terminates at a pass of electrodes 204. The electrodes 204 are connected to a console 206 that contains a modulation circuit. The wire functions as an antennae to emit RF energy that is absorbed by tissue placed in the petri dish 202.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A medical method for treating an organic substance, comprising:
applying a broad spectrum of radio frequency energy to the organic substance.

2. The method of claim 1, wherein the broad spectrum ranges between 50 kilohertz and 200 megahertz.

3. The method of claim 1, wherein the radio frequency energy is applied with electro-magnetic waves.

4. The method of claim 1, wherein the radio frequency energy is applied with electrical current passing through the tissue.

5. The method of claim 1, wherein the broad spectrum of radio frequency energy is applied as a sequence of signals each having a different frequency.

6. The method of claim 1, wherein the radio frequency energy is a noise spectrum.

7. An apparatus for applying a broad spectrum of radio frequency energy to an organic substance, comprising:
a transmitter that is coupled to the organic substance; and,
a circuit connected to said transmitter, said circuit generates a broad spectrum of radio frequency energy.

8. The apparatus of claim 7, wherein said transmitter includes an electrode.

9. The apparatus of claim 7, wherein said transmitter includes an antenna.

10. The apparatus of claim 7, wherein the broad spectrum ranges between 50 kilohertz and 200 megahertz.

11. The apparatus of claim 7, wherein said control circuit sequentially generates a series of signals that each have a different frequency.

12. The apparatus of claim 7, wherein the radio frequency energy is a noise spectrum.

13. The apparatus of claim 7, wherein said control circuit includes a controller connected to a digital to analog converter, said digital to analog converter being connected to said transmitter.

14. A medical method for enhancing mitosis in a cell, comprising:
applying a broad spectrum of radio frequency energy to the cell.

15. The method of claim 14, wherein the broad spectrum ranges between 50 kilohertz and 200 megahertz.

16. The method of claim 14, wherein the radio frequency energy is applied with electro-magnetic waves.

17. The method of claim 14, wherein the radio frequency energy is applied with electrical current passing through the tissue.

18. The method of claim 14, wherein the broad spectrum of radio frequency energy is applied as a sequence of signals each having a different frequency.

19. The method of claim 14, wherein the radio frequency energy is a noise spectrum.

20. A medical method, comprising:
implanting a medical device into a patient; and,
transmitting a broad spectrum of radio frequency energy to the medical device.

21. The method of claim 20, wherein the broad spectrum ranges between 50 kilohertz and 200 megahertz.

22. The method of claim 20, wherein the radio frequency energy is applied with electro-magnetic waves.

23. The method of claim 20, wherein the broad spectrum of radio frequency energy is applied as a sequence of signals each having a different frequency.

24. The method of claim 20, wherein the radio frequency energy is a noise spectrum.

25. A medical method for inducing beta-endorphin production, comprising:
applying a broad spectrum of radio frequency energy to at least one cell.

26. The method of claim 25, wherein the broad spectrum ranges between 50 kilohertz and 200 megahertz.

27. The method of claim 25, wherein the radio frequency energy is applied with electro-magnetic waves.

28. The method of claim 25, wherein the radio frequency energy is applied with electrical current passing through the tissue.

29. The method of claim 25, wherein the broad spectrum of radio frequency energy is applied as a sequence of signals each having a different frequency.

30. The method of claim 25, wherein the radio frequency energy is a noise spectrum.

31. The method of claim 25, wherein the cell is a stem cell.
